(19) United States
(12) Patent Application Publication
(10) Pub. No.: US 2025/0099750 A1
(43) Pub. Date: Mar. 27, 2025

(54) IONTOPHORESIS TRANSDERMAL DRUG DELIVERY DEVICE AND SYSTEM FOR CANCER TREATMENT

(71) Applicant: SOGANG UNIVERSITY RESEARCH FOUNDATION, Seoul (KR)

(72) Inventors: Bong Geun CHUNG, Seoul (KR); Tae Hyeon Kim, Seoul (KR); Na Yeon Kim, Seoul (KR); Hee Uk Lee, Seoul (KR); Ji Wook Choi, Seoul (KR)

(21) Appl. No.: 18/892,772

(22) Filed: Sep. 23, 2024

(30) Foreign Application Priority Data
Sep. 25, 2023 (KR) 10-2023-0128426

(51) Int. Cl. A61N 1/30; A61N 1/08

(52) U.S. Cl. CPC A61N 1/303; A61N 1/08

(57) ABSTRACT

Disclosed is an iontophoresis transdermal drug delivery device for cancer treatment, the device including: a power module including a battery; a microcontroller module configured to receive a voltage from the power module to output a constant-current electrical signal; and a microfluidic chip configured to store a drug and deliver the stored drug to the skin on the basis of the electrical signal.

### CROSS-REFERENCE TO RELATED APPLICATION

This application is based on and claims priority under 35 U.S.C. 119 to Korean Patent Application No. 10-2023-0128426, filed on Sep. 25, 2023, in the Korean Intellectual Property Office, the disclosure of which is herein incorporated by reference in its entirety.

### FIELD

The present disclosure relates to an iontophoresis transdermal drug delivery device and system for cancer treatment, both of which promote transdermal drug delivery by using low-intensity currents.

The present disclosure has been made with the support of the ministry of Science and ICT under Project ID. No. 1711181531, and sub-project No. 2022R1A2C2003724, which was conducted by Sogang University in the research program named “Individual Basic Research (Ministry of Science and ICT)” as a branch of the research project titled “Development of functional electrode chip and nanomaterial for iPSC-derived neural differentiation” under the research management of National Research Foundation of Korea, from Mar. 1, 2023 to Feb. 29, 2024.

The present disclosure has been made with the support of the ministry of Science and ICT, ministry of Health and Welfare under Project ID. No. 1711179437, and sub-project No. 00070316, which was conducted by Sogang University, Seoul National University in the research program named “Pan-Ministerial Regenerative Medicine Technology Development Project” as a branch of the research project titled “Detection and removal of culture adapted human pluripotent stem cells with genetic aberration for safety assurance” under the research management of Pan-Ministerial Integrated R&D Support, from Jan. 1,2023 to Dec. 31, 2023.

The present disclosure has been made with the support of the ministry of Science and ICT under Project ID. No. 1711198532, and sub-project No. RS-2023-00259341, which was conducted by Sogang University in the research program named “Collaboration Hub Construction Project for Excellent Overseas Research Institution” as a branch of the research project titled “Sogang-UPenn Convergence Research Center for Theranostics of Emerging Infectious Disease” under the research management of National Research Foundation of Korea, from Jul. 1, 2023 to Dec. 31, 2023.

The present disclosure has been made with the support of the Ministry of Education under Project ID. No. 1345312114, and sub-project No. 2016R1A6A1A03012845, which was conducted by Sogang University in the research program named “Basic research Program” as a branch of the research project titled “Development of Nanobiochip Platform to Analyze Drug Evaluation in Brain Disease” under the research management of National Research Foundation of Korea, from Jan. 1, 2023 to Dec. 31, 2023.

### BACKGROUND

Global statistics in 2020 predicted there would be nearly 20 million new cancer cases and 10 million deaths worldwide. However, conventional cancer treatments, such as intravenous chemotherapy or oral medication, have limitations in view of drug efficacy and patient compliance.

For instance, conventional cancer treatments may cause issues, such as systemic side effects, low accessibility to tumor site, and frequent hospital visits required to ensure effective cancer treatment. In particular, conventional cancer treatments distribute drugs indiscriminately throughout the body, increasing the risk of damaging healthy tissues.

Moreover, conventional cancer treatments may cause limited treatment effects due to low accessibility to tumor site, and thus larger amounts of drugs are often administered to deliver sufficient concentrations of drugs to target sites.

To address these limitations, devices and systems capable of delivering drugs through transdermal permeation are recently needed. However, the transdermal route is challenging due to limited drug permeation caused by the skin barrier formed by the stratum corneum.

The description in this background section is provided merely to promote understanding of the background of the present disclosure and may include contents that are not already known to those of ordinary skill in the art to which this disclosure belongs.

### SUMMARY

An aspect of the present disclosure is to provide an iontophoresis transdermal drug delivery device and system for cancer treatment, whereby precise and localized drug delivery can be achieved through the iontophoresis-based drug delivery that is remotely controlled by a mobile application.

In accordance with an aspect of the present disclosure, there is provided an iontophoresis transdermal drug delivery device for cancer treatment, the device including: a power module including a battery; a microcontroller module configured to receive a voltage from the power module to output a constant-current electrical signal; and a microfluidic chip configured to store a drug and deliver the stored drug to the skin on the basis of the electrical signal.

The microfluidic chip may be composed of a polymethylmethacrylate (PMMA)-based fluidic plastic.

The microfluidic chip may include a drug source, a membrane, and a receptor channel.

The membrane may be disposed between the drug source and the receptor channel.

In the microfluidic chip, the drug source and the membrane may be fixed by a polymethylsiloxane (PDMS) having adhesive properties with an elastomeric polymer and a curing agent mixed at a first ratio.

In the microfluidic chip, the drug source, the membrane, and the receptor channel may be fixed to a PMMA plate by curing a polymethylsiloxane (PDMS) with an elastomeric polymer and a curing agent mixed at a second ratio around the drug source, the membrane, and the receptor channel, to prevent the leakage of a drug between the dug source, the membrane, and the receptor channel.

In accordance with another aspect of the present disclosure, there is provided an iontophoresis transdermal drug delivery system for cancer treatment, the system including: a drug delivery device configured to store a drug and generate an electrical signal according to an iontophoresis mode to deliver the stored drug to the skin; and a mobile terminal configured to generate a control signal for switching the iontophoresis mode or controlling the intensity of the electrical signal and transmit the control signal to the drug delivery device to control the drug delivery to the skin.

The mobile terminal may switch the polarity of the electrical signal according to the control signal.

The iontophoresis mode may include a first mode for increasing drug permeation to the skin and a second mode for interfering with drug permeation to the skin.

When the polarity of the drug is anionic, the mobile terminal may switch the first mode to an anodic mode where the polarity of the electrical signal is anodic, and switch the second mode to a cathodic mode where the polarity of the electrical signal is cathodic.

When the polarity of the drug is cationic, the mobile terminal may switch the first mode to a cathodic mode where the polarity of the electrical signal is cathodic, and switch the second mode to an anodic mode where the polarity of the electrical signal is anodic.

According to the preset disclosure, an environment capable of offering precise and localized drug delivery can be provided through an iontophoresis-based transdermal drug delivery system that is remotely controlled by a mobile application.

Furthermore, the present disclosure can reduce the risk of systemic side effects by localized drug delivery and offer an environment capable of enhancing the treatment effect by increasing the accessibility to tumor cells.

Furthermore, the present disclosure can immediately terminate drug administration when symptoms associated with side effects occur, and thus offer an environment capable of minimizing the damage from side effects compared with the oral and injectable administration manners that cannot be reversed once injected.

Furthermore, unlike the conventional injectable manner, the transdermal permeation manner of the present disclosure does not require professional medical personnel and thus offers an environment capable of increasing patient compliance by reducing hospital visits.

Furthermore, the iontophoresis transdermal drug delivery system for cancer treatment according to the present disclosure can be a revolutionary tool for self-administration and personalized medical platforms for cancer treatment and offers an environment that can be applied not only to cancer treatment but also to various chronic diseases, such as diabetes and heart disease.

### BRIEF DESCRIPTION OF THE DRAWINGS

The above and other aspects, features and advantages of the present disclosure will be more apparent from the following detailed description taken in conjunction with the accompanying drawings.

FIG. 1 is a block diagram of an iontophoresis transdermal drug delivery device for cancer treatment according to an embodiment of the present disclosure.

FIG. 2 is a schematic diagram of an iontophoresis transdermal drug delivery device according to an embodiment of the present disclosure.

FIG. 3 illustrates the results of numerical simulation analysis for the mechanism of iontophoresis transdermal drug delivery according to an embodiment of the present disclosure.

FIG. 4 illustrates the results of drug permeation analysis according to an embodiment of the present disclosure.

FIG. 5 illustrates the analysis results of in vitro cell experiments according to an embodiment of the present disclosure.

FIG. 6 illustrates the drug delivery mechanism of the iontophoresis transdermal drug delivery system according to an embodiment of the present disclosure and the results of performance evaluation thereof.

### DETAILED DESCRIPTION

Hereinafter, exemplary embodiments of the present disclosure will be described in detail with reference to the accompanying drawings so that persons having with ordinary skill in the art to which the present disclosure pertains can easily carry out the present disclosure. However, the present disclosure may be embodied in various different forms, and therefore is not limited to embodiments to be described herein. In the drawings, to clearly describe the present disclosure, portions which are not related to the description of the present disclosure will be omitted and similar portions are denoted by similar reference numerals throughout the specification.

Throughout the specification, when a certain portion “comprises”, “contains”, or “includes” a certain component, this means that the certain portion may further comprise, contain, or include other components, rather than excluding the other components, unless otherwise specifically stated. The term “ . . . unit”, “ . . . er”, or “ . . . module” used herein refers to a unit that performs at least one function or operation, and this may be implemented in hardware, software, or a combination thereof.

Various embodiments are now described with reference to the drawings. Herein, various descriptions are suggested to provide understanding of the present disclosure. However, it is obvious that the exemplary embodiments may be embodied without even specific description.

Terminologies, such as “component”, “module”, or “system” used herein indicate a computer-related entity, hardware, firmware, software, a combination of software and hardware, or execution of software. For example, a component may be a procedure which is executed in a processor, a processor, an object, an execution thread, a program, and/or a computer, but is not limited thereto. For example, both an application which is executed in a computing device and a computing device may be components. One or more components may be stayed within the processor and/or execution thread. One component may be localized in one computer. One component may be distributed between two or more computers. Such components may be executed from various computer readable media having various data structures stored therein. The components may communicate with each other through local and/or remote processings in accordance with a signal (for example, data transmitted through other system and a network such as Internet through data and/or a signal from one component which interacts with other component in a local system or a distributed system) having one or more data packets.

Additionally, the term “or” is intended to refer to not exclusive “or”, but inclusive “or”. That is, when it is not specified or unclear on the context, “X uses A or B” is intended to mean one of natural inclusive substitutions. That is, when X uses A; X uses B; or X uses both A and B, “X uses A or B” may be applied to any of the above instances. Further, it should be understood that the term “and/or” used in this specification designates and includes all available combinations of one or more items among listed related items.

The term “comprise”, “includes”, “contains” and/or “comprising”, “including”, or “containing” is understood that the corresponding feature and/or component are present. However, it should be understood that the term “comprise”, “include”, “contain” and/or “comprising”, “including”, or “containing” does not preclude existence or addition of one or more other features, constituent elements and/or these groups. Further, when it is not separately specified or it is not clear from the context to indicate a singular form, the singular form in the specification and the claims is generally interpreted to represent “one or more”.

In addition, the term “at least one of A or B” should be interpreted to mean “a case including only A”, “a case including only B”, and “a case in which A and B are combined”.

Those skilled in the art will further appreciate that the various illustrative logical blocks, configurations, modules, circuits, means, logics, and algorithm steps described in connection with the embodiments disclosed herein may be implemented as electronic hardware, computer software, or combinations thereof. To clearly illustrate the interchangeability of hardware and software, various illustrative components, blocks, configurations, means, logics, modules, circuits, and steps have been described above generally in terms of their functionality. Whether the functionality is implemented as hardware or software depends on a specific application and design constraints imposed on the overall system. Those skilled in the art may implement the described functionality in various ways for each of specific applications. However, decisions of such implementations should be interpreted without departing from the scope of the present disclosure.

Description of the suggested exemplary embodiments is provided to allow those skilled in the art to use or embody the present disclosure. Various modifications to these embodiments may be apparent to those skilled in the art. Generic principles defined herein may be applied to other embodiments without departing from the scope of the present disclosure. Therefore, the present disclosure is not limited to the embodiments suggested herein. The present disclosure needs to be interpreted within the broadest scope consistent with principles suggested herein and novel features.

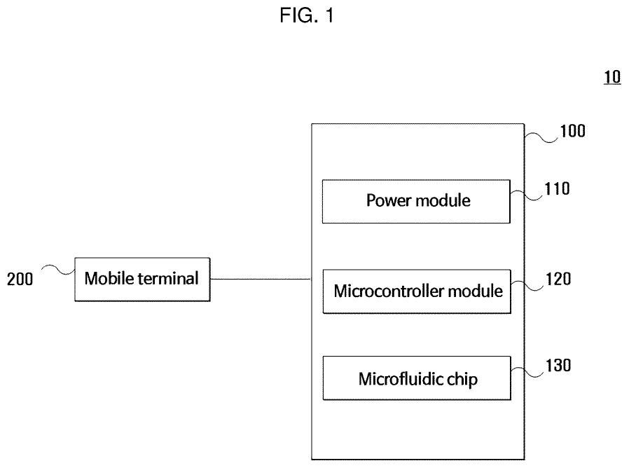

Hereinafter, an iontophoresis transdermal drug delivery device and system for cancer treatment according to an embodiment of the present disclosure is described in detail with reference to FIGS. 1 to 6.

FIG. 1 is a block diagram of an iontophoresis transdermal drug delivery system for cancer treatment according to an embodiment of the present disclosure.

An iontophoresis transdermal drug delivery system 10 for cancer treatment according to an embodiment of the present disclosure can minimize systemic side effects through localized drug delivery by an iontophoresis method that promotes the transdermal drug delivery using a low-intensity current. The iontophoresis transdermal drug delivery system 10 for cancer treatment according to an embodiment of the present disclosure was modeled through finite element analysis, and the performance thereof was verified through skin permeation tests using a plastic-based microfluidic chip.

Additionally, the iontophoresis transdermal drug delivery system 10 for cancer treatment according to an embodiment of the present disclosure was demonstrated to have significantly improved drug permeability for cancer treatment, through the in vitro cell test and skin deposition test. Therefore, the iontophoresis transdermal drug delivery system 10 for cancer treatment according to an embodiment of the present disclosure can be utilized as a platform useful for self-administered cancer treatments.

Referring to FIG. 1, the iontophoresis transdermal drug delivery system 10 for cancer treatment according to an embodiment of the present disclosure may include an iontophoresis transdermal drug delivery device 100 and a mobile terminal 200.

The iontophoresis transdermal drug delivery device 100 may store a drug and generate an electrical signal according to an iontophoresis mode to deliver the stored drug to the skin.

The iontophoresis transdermal drug delivery device 100 may include: a power module 110 including a battery; a microcontroller module 120 configured to receive a voltage from the power module 110 to output a constant-current electrical signal; and a microfluidic chip 130 configured to store a drug and deliver the stored drug to the skin on the basis of the electrical signal.

The microfluidic chip 130 may be composed of a polymethylmethacrylate (PMMA)-based fluidic plastic.

The microfluidic chip 130 may include a drug source, a membrane, and a receptor channel. The drug source, the membrane, and the receptor channel may be disposed above a tetragonal PMMA plate. The membrane may be disposed between the drug source and the receptor channel.

In the microfluidic chip 130, the drug source and the membrane may be fixed with a PDMS ring inserted between the drug source and the membrane. For example, in the microfluidic chip 130, the drug source and the membrane may be fixed by a polymethylsiloxane (PDMS) having adhesive properties with an elastomeric polymer and a curing agent mixed at a first ratio (e.g., 40:1).

Alternatively, in the microfluidic chip 130, the drug source, the membrane, and the receptor channel may be fixed to the PMMA plate by curing a polymethylsiloxane (PDMS) with an elastomeric polymer and a curing agent mixed at a second ratio (e.g., 10:1) around the drug source, the membrane, and the receptor channel, so as to prevent the leakage of a drug between the dug source, the membrane, and the receptor channel.

The mobile terminal 200 may generate a control signal for switching the iontophoresis mode or controlling the intensity of the electrical signal, and control the amount of a drug delivered to the skin through the control signal. The mobile terminal 200 may transmit the control signal to the drug delivery device to control the drug delivery to the skin.

For example, the mobile terminal 200 may switch the polarity of the electrical signal according to the control signal. In such a case, the iontophoresis mode may include a first mode for increasing drug permeation to the skin and a second mode for hindering drug permeation to the skin.

When the polarity of the drug is anionic, the mobile terminal 200 may switch the first mode to an anodic mode where the polarity of the electrical signal is anodic, and switch the second mode to a cathodic mode where the polarity of the electrical signal is cathodic.

When the polarity of the drug is cationic, the mobile terminal 200 may switch the first mode to a cathodic mode where the polarity of the electrical signal is cathodic, and switch the second mode to an anodic mode where the polarity of the electrical signal is anodic.

The iontophoresis transdermal drug delivery system 10 for cancer treatment according to an embodiment of the present disclosure can allow charged drug molecules to permeate through the skin by using a low-intensity current and control the permeation of the drug by switching the charge of a working electrode.

The principal mechanisms according to the present disclosure are electro-repulsion and electro-osmosis. The electro-repulsion refers to the movement of charged drugs driven by an electric field, and the drug permeation may be achieved by electro-repulsion when the same polarity as the drug is applied to the electrode. The electro-osmosis involves the bulk movement of a fluid carrying a charged or neutral drug, and this facilitates the fluid flow in the skin to help the drug to be delivered more efficiently. The iontophoresis transdermal drug delivery system 10 for cancer treatment according to an embodiment of the present disclosure promotes the drug delivery on the basis of an electrical signal, and thus can be utilized for remote drug control through a mobile application.

Furthermore, the iontophoresis transdermal drug delivery system 10 for cancer treatment according to an embodiment of the present disclosure can enhance the drug permeation and sustain the enhancement effect even after the ending of the drug permeation. Furthermore, the iontophoresis transdermal drug delivery system 10 for cancer treatment according to an embodiment of the present disclosure can control the drug permeation by switching the charge of the working electrode during the application of iontophoresis.

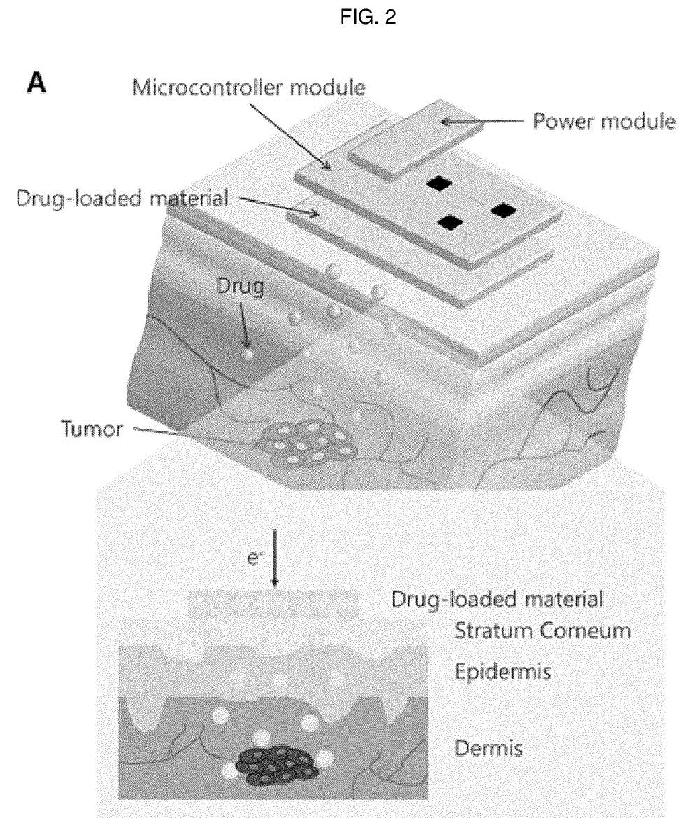

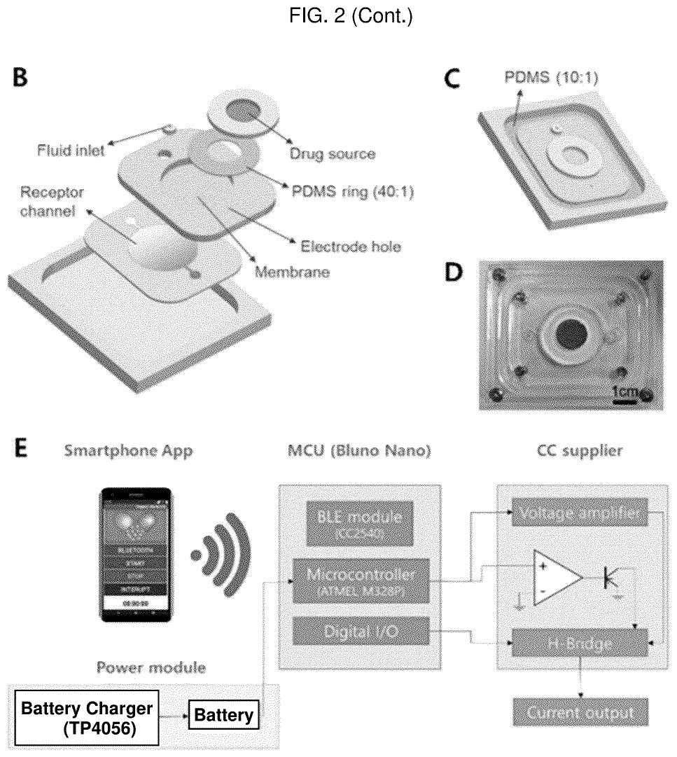

FIG. 2 is a schematic diagram of the iontophoresis transdermal drug delivery device with respect to the mechanism of iontophoresis transdermal drug delivery according to an embodiment of the present disclosure.

Referring to FIG. 2, an iontophoresis transdermal drug delivery device according to an embodiment of the present disclosure may be designed to emulate the mechanism of skin permeation by inserting a membrane between a receptor channel and a drug source. Specifically, FIG. 2A illustrates an example of cancer treatment through an iontophoresis-based transdermal drug delivery system (TDDS); FIG. 2B is an exploded diagram of a microfluidic chip according to an embodiment of the present disclosure; FIG. 2C is an assembly diagram of the microfluidic chip according to an embodiment of the present disclosure; FIG. 2D is an embodiment of a fabricated microfluidic chip; and FIG. 2E is a block diagram of an electronic control system for iontophoresis transdermal drug delivery using a smartphone.

A power module, a microcontroller module, and a drug-loaded material shown in FIG. 2A may be the power module 110, the microcontroller module 120, and the microfluidic chip 130 of the iontophoresis transdermal drug delivery device 100 according to the present disclosure, respectively.

Referring to FIG. 2B, the drug source may have a hollow shape with an inner diameter of 12 mm and a height of 5 mm, resulting in a diffusion area of 1.131 cm². The height of the receptor channel may be 1 mm, and the chamber may have a diameter of 20 mm.

A skin-mimicking membrane (e.g., Strat-M) may be inserted between the drug source and the receptor channel. The drug source and the membrane may be fixed by a PDMS having adhesive properties with an elastomeric polymer and a curing agent mixed at a ratio of 40:1.

The fabrication of the receptor channel with the PMMA plate may cause a fluidic leakage, and simply screwing the PMMA plate may not be sufficient to prevent the fluid from escaping between PMMA layers. In the present disclosure, the PDMS around the PMMA plate was cured to solve the fluid leakage issue, as shown in FIG. 2C. For example, in order to prevent the fluid leakage between the several PMMA layers connected in the microfluidic chip 130, PDMS with a ratio of 10:1 may be cured to fix the drug source, the membrane, and the receptor channel on the PMMA plate.

In the present disclosure, to ensure stable drug loading, a PDMS ring having adhesive properties may be disposed between the drug source and the membrane as shown in FIG. 2D. The adhesive strength and Young's modulus of PDMS may vary depending on the mixing ratio of the pre-polymer and the curing agent. In general, the adhesion efficiency tends to increase according to the concentration of the pre-polymer over the curing agent in PDMS. Excessive pre-polymer with a high mixing ratio may lead to a high density of silanol groups on the surface due to incomplete cross-linking, enhancing the interaction with other polar materials and inducing strong adhesive strength. However, a high mixing ratio may cause the material to tear easily due to low Young's modulus. For example, a mixing ratio exceeding 70:1 may lead to a highly viscous liquid due to the insufficient cross-linking. A mixing ratio of 50:1 or 60:1 may result in a low Young's modulus. Alternatively, a relatively low mixing ratio being 20:1 or 30:1 may not be suitable for the PMMA plate adhesion, and thus in the present disclosure, the mixing ratio for the PDMS ring applicable to wearable devices was optimized to be 40:1. Additionally, as shown in FIG. 2E, the drug delivery to the skin can be controlled through wireless control using a mobile terminal, such as a smartphone.

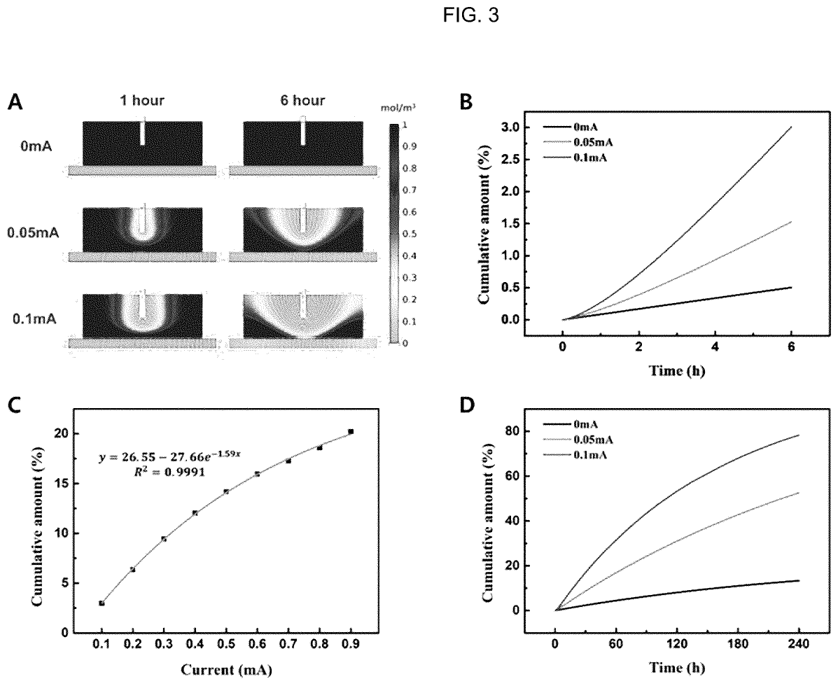

FIG. 3 shows the results of numerical simulation analysis according to an embodiment of the present disclosure.

FIG. 3 illustrates the numerical simulation to elucidate the effect of iontophoresis on the iontophoresis transdermal drug delivery system (TDDS) of the present disclosure. To investigate the drug permeation behavior of iontophoresis transdermal administration of Dox, finite element analysis (FEA)-based theoretical simulations were performed. Specifically, FIG. 3A shows snapshots comparing the transient drug concentration under 0, 0.05, and 0.1 mA at 1 and 6 h; FIG. 3B shows the comparison results of cumulative amounts of permeated drugs between passive diffusion and iontophoresis; FIG. 3C shows the regression analysis results of cumulative amounts over current intensities; and FIG. 3D shows cumulative amounts over a long-term duration.

Referring to FIG. 3A and 3B, the numerical simulations provide the effect of current intensity on drug permeation through the skin, and the application of iontophoresis significantly enhanced drug permeation, indicating that iontophoresis can promote drug delivery through the skin.

For example, the cationic drug, Dox, was repelled by the anodic electrode and moved closer to the membrane (FIG. 3A). Particularly, the only factor affecting drug transport was the current intensity, which was corroborated with consistent results despite varying flow rates in the receptor channel and different electrical conductivities of the drug source.

Referring to FIG. 3B, the results based on current intensity demonstrate that the degree of enhancement is directly proportional to the applied current intensity. For example, compared with the passive condition (0 mA), the iontophoresis at current intensities of 0.05 mA and 0.1 mA resulted in 3- and 6-fold higher permeation fluxes, respectively, after 6 h. However, an increase of the current intensity above 0.1 mA may cause potential skin irritation. Therefore, a current intensity as high as 0.1 mA or higher should be considered as the upper limit for safe and effective application, and it may be important to carefully select an appropriate current intensity to achieve the desired drug permeation while considering patient safety.

Referring to FIG. 3C, the cumulative amount of the drug permeated after 6 h was found to be correlated with the current intensity. The regression equation for this correlation was determined as y=26.55−27.66e−1.59x with R2 of 0.9991. Referring to FIG. 3D, extending the duration to 240 h (10 days) reveals that the effect of electrical stimulation on permeation flux can remain consistently stable.

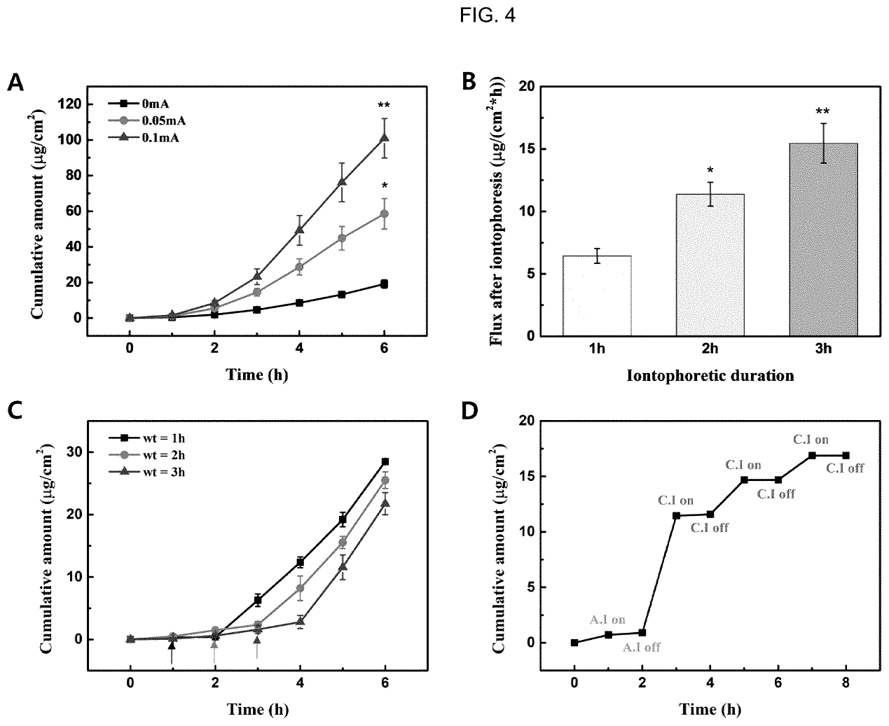

FIG. 4 illustrates the results of drug permeation analysis according to an embodiment of the present disclosure.

Specifically, FIG. 4A shows the comparison results of cumulative amounts of permeated drugs between passive diffusion and iontophoresis; FIG. 4B shows the comparison results of the permeation flux over the iontophoresis duration; FIG. 4C shows the triggering effect of iontophoresis; and FIG. 4D shows an example of controllable drug permeation by alternating the iontophoresis mode.

The experimental results obtained from the skin permeation test were similar to the numerical simulation results (FIG. 4A and FIG. 3B). The iontophoresis with current intensities of 0.05 mA and 0.1 mA resulted in 3- and 5.3-fold permeation fluxes, respectively, compared with the passive condition, after 6 h. Especially, the enhanced permeation flux persisted after the termination of iontophoresis, and this continued enhancement was directly proportional to the iontophoresis duration (FIG. 4B). This phenomenon is attributed to structural rupture of the membrane, potentially expanding the drug delivery pathway. The permeation fluxes showed significant differences with respect to iontophoresis, indicating that iontophoresis significantly increased the permeation flux (FIG. 4C). The enhancement in drug permeation rate may not be immediately observed but may begin after 1 h. This delay is attributed to electrical stimulation that can induce a chemical reaction and change the pH value of the drug source. Since the concentration of ionized drugs is affected by pH change, this change may interfere with drug permeation during iontophoresis process. The waiting time (wt) before applying iontophoresis did not significantly affect the permeation flux.

Previous studies mainly focused on increasing drug delivery rates rather than achieving accurate and rapid termination, but the present disclosure can offer the advantage of accurate and rapid termination by alternating the polarity of the working electrode to act as an on/off switch (FIG. 4D). In the present disclosure, the cathodic iontophoresis significantly interferes with the permeation of Dox, which is a drug having the positive polarity, whereas anodic iontophoresis can enhance the permeation of Dox. Therefore, when symptoms associated with side effects (e.g., hyperthermia) occur, the present disclosure can offer an environment capable of rapidly resolving side effects by immediately terminating drug delivery. Furthermore, the present disclosure can reduce side effects and ensure optimal treatment results.

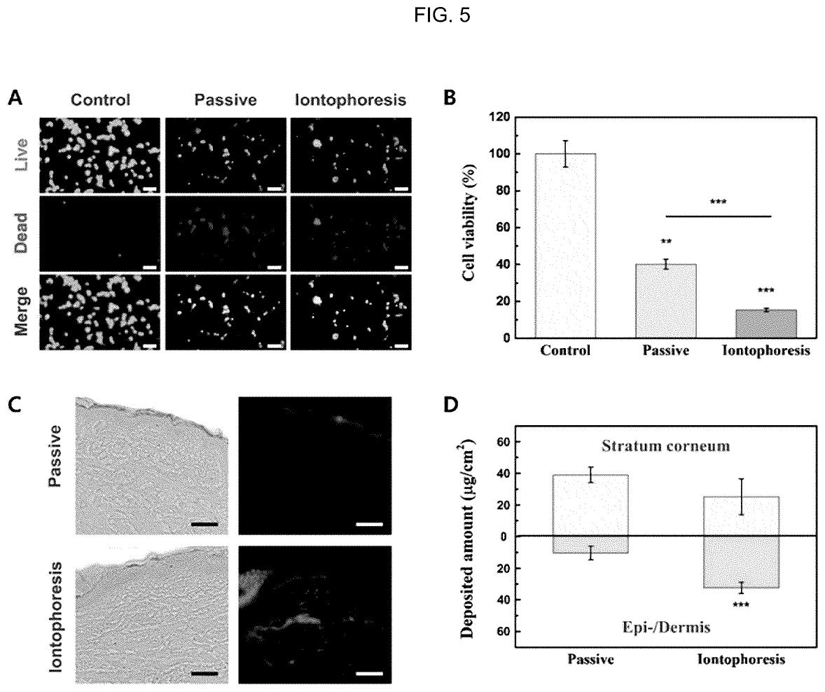

FIG. 5 illustrates the analysis results of in vitro cell experiments according to an embodiment of the present disclosure.

Referring to FIG. 5, the efficacy of the iontophoresis transdermal drug delivery system of the present disclosure was evaluated through the in vitro cell test and skin deposition test.

Specifically, FIG. 5A shows the live/dead analysis results according to the control, passive, and iontophoresis; FIG. 5A illustrates the comparison results of cell viability under three conditions of control, passive, and iontophoresis; FIG. 5C shows optical and fluorescent images from histological examination; and FIG. 5D shows the deposited amounts of drugs within the stratum corneum and epidermis/dermis, respectively.

In the present disclosure, the effect of iontophoresis on the cancer cell fate was evaluated on the basis of the results from numerical simulations and skin permeation tests, and live/dead analysis and MTT analysis were performed to demonstrate that the viability of cancer cells was significantly lower in the iontophoresis condition compared with the control and passive conditions (FIGS. 5A and 5B). The cell viability was measured to be 100%, 40.2%, and 15.4% in the control, passive, and iontophoresis group, respectively. These results indicate that the iontophoresis of the present disclosure significantly enhanced the delivery of Dox through the skin and provided a superior anticancer effect. That is, the iontophoresis-controlled and targeted drug delivery can lead to higher drug concentrations at the desired tumor sites, enhancing cancer treatment efficacy. Furthermore, the skin deposition test was performed to determine the depth of drug permeation into the porcine skin (FIGS. 5C and 5D). Under passive diffusion, Dox remained predominantly on the stratum corneum, an outermost layer of the skin, without appreciable permeation into the deeper layers of the epidermis and dermis. This limited drug permeation restricts the entry of external hydrophilic molecules due to the barrier property of the stratum corneum. However, Dox deeply permeated into the epidermis and dermis layers through iontophoresis, effectively crossing through the stratum corneum barrier. The epidermal and dermal permeation through iontophoresis increased by 3.1-fold compared with the passive diffusion (FIG. 5D). The results of the skin deposition test demonstrated that this enhanced drug permeation overcomes the skin barrier and is effectively applied to the cancer treatment through the deep permeation into the epidermis and dermis layers.

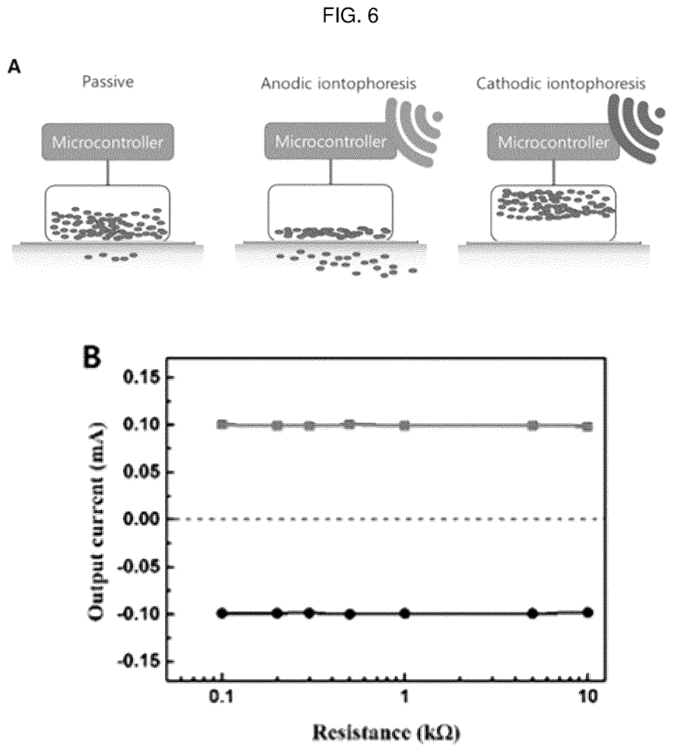

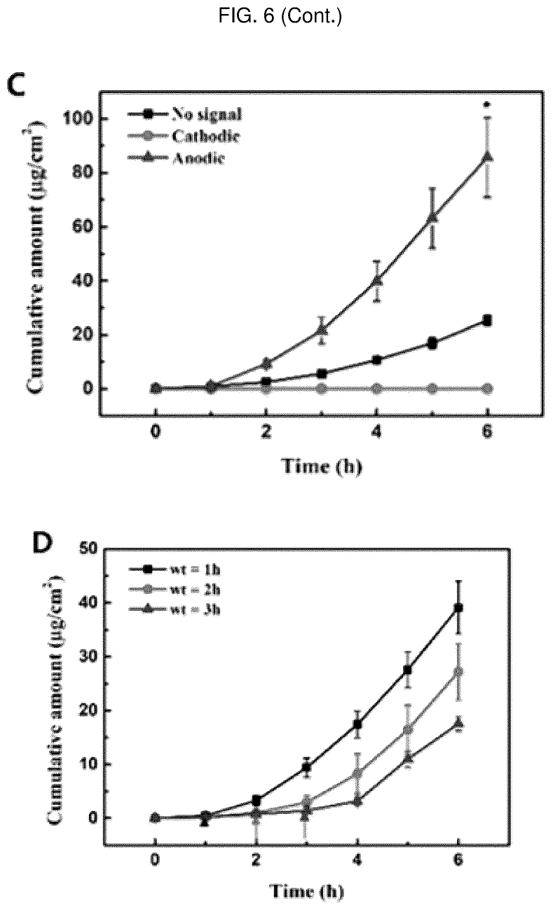

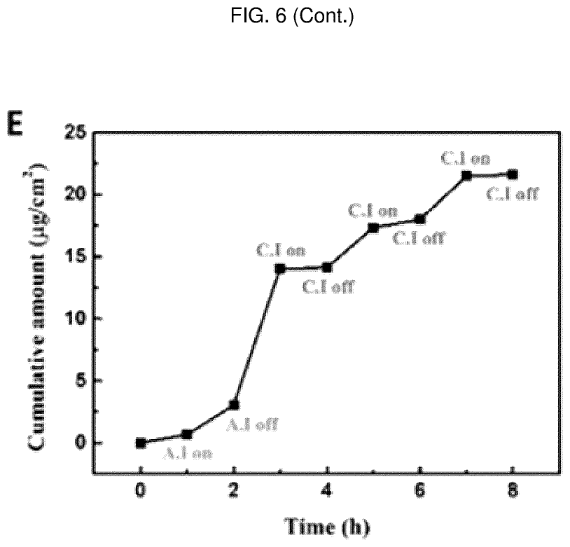

FIG. 6 illustrates the drug delivery mechanism of the iontophoresis transdermal drug delivery system according to an embodiment of the present disclosure and the results of performance evaluation thereof.

FIG. 6 illustrates the evaluation of remote-control performance of the iontophoresis transdermal drug delivery system. Specifically, FIG. 6A shows drug delivery mechanisms for iontophoresis modes; FIG. 6B shows the performance evaluations for the output current from the microcontroller module; FIG. 6C shows the comparison results of cumulative amounts according to the Bluetooth signal; FIG. 6D shows a triggering effect of iontophoresis; and FIG. 6E shows the results of controlling drug permeation by alternating iontophoresis modes.

Referring to FIG. 6A, the drug permeation to the skin can be controlled according to passive, anodic iontophoresis, and cathodic iontophoresis modes.

The iontophoresis modes may be selected by the transmission of Bluetooth signals through the mobile terminal, and if necessary, the drug delivery may be enhanced or interrupted. The present disclosure may include a smartphone application capable of transmitting Bluetooth signals to the microcontroller module (MCU of a PCB module), which outputs the constant current (CC). To selectively activate the iontophoresis mode (anodic or cathodic), the microcontroller module of the present disclosure may include a CC supplier including the H-bridge circuit.

The stability of this constant current supply may be crucial for protecting the skin against skin irritation. The evaluation results of the current output from the PCB revealed consistent performance up to 10 kΩ, regardless of external resistances (FIG. 6B).

For instance, according to the Ohm's law (V=IR), the constant current (CC) circuit reduces voltage as the skin resistance diminishes during iontophoresis, whereas the constant voltage (CV) circuit increases current as the skin resistance decreases, potentially causing skin irritation. Thus, the present disclosure dramatically adapts to variations in skin resistance, ensuring safe and stable drug permeation. The skin permeation test was performed using an electronic system controlled by a smartphone application (FIGS. 6C to 6E). Anodic iontophoresis exhibited a higher cumulative amount of permeated Dox compared with no signals, whereas cathodic iontophoresis did not permit permeation (FIG. 6C). The present disclosure shows that through the smartphone application, the permeation of Dox can be controlled by alternating iontophoresis modes (FIG. 6E).

As set forth above, the iontophoresis transdermal drug delivery device and system for cancer treatment according to an embodiment of the present disclosure offer an environment capable of providing precise and localized drug delivery through an iontophoresis-based transdermal drug delivery system remotely controlled by a mobile application.

Furthermore, the present disclosure can reduce the risk of systemic side effects by localized drug delivery and offer an environment capable of enhancing the treatment effect by increasing the accessibility to tumor cells.

Furthermore, the present disclosure can immediately terminate drug administration when symptoms associated with side effects occur, and thus offer an environment capable of minimizing the damage from side effects compared with the oral and injectable administration manners that cannot be reversed once injected.

Furthermore, unlike the conventional injectable manner, the transdermal permeation manner of the present disclosure does not require professional medical personnel and thus offers an environment capable of increasing patient compliance by reducing hospital visits.

Furthermore, the iontophoresis transdermal drug delivery system for cancer treatment according to the present disclosure can be a revolutionary tool for self-administration and personalized medical platforms for cancer treatment and offers an environment that can be applied not only to cancer treatment but also to various chronic diseases, such as diabetes and heart disease.

The embodiments of the present disclosure as described above are not implemented only through the device and method and may be implemented through programs that realize functions corresponding to the configurations of the embodiments of the present disclosure or recording media having the programs sorted thereon. Such recording media may be implemented on a user terminal as well as a server.

Although the embodiments of the present disclosure have been described in detail, the scope of the present disclosure is not limited thereto, and various changes and modifications made by those skilled in the art using the basic concept of the present disclosure defined in the following claims also fall within the scope of the present disclosure.

What is claimed is:

1. An iontophoresis transdermal drug delivery device for cancer treatment, the device comprising:
a power module comprising a battery;
a microcontroller module configured to receive a voltage from the power module to output a constant-current electrical signal; and
a microfluidic chip configured to store a drug and deliver the stored drug to the skin on the basis of the electrical signal.

2. The iontophoresis transdermal drug delivery device of claim 1, wherein the microfluidic chip is composed of a polymethylmethacrylate (PMMA)-based fluidic plastic.

3. The iontophoresis transdermal drug delivery device of claim 1, wherein the microfluidic chip comprises a drug source, a membrane, and a receptor channel.

4. The iontophoresis transdermal drug delivery device of claim 3, wherein the membrane is disposed between the drug source and the receptor channel.

5. The iontophoresis transdermal drug delivery device of claim 3, wherein in the microfluidic chip, the drug source and the membrane are fixed by a polymethylsiloxane (PDMS) having adhesive properties with an elastomeric polymer and a curing agent mixed at a first ratio.

6. The iontophoresis transdermal drug delivery device of claim 3, wherein in the microfluidic chip, the drug source, the membrane, and the receptor channel are fixed to a PMMA plate by curing a polymethylsiloxane (PDMS) with an elastomeric polymer and a curing agent mixed at a second ratio around the drug source, the membrane, and the receptor channel, to prevent the leakage of a drug between the dug source, the membrane, and the receptor channel.

7. An iontophoresis transdermal drug delivery system for cancer treatment, the system comprising:
a drug delivery device configured to store a drug and generate an electrical signal according to an iontophoresis mode to deliver the stored drug to the skin; and
a mobile terminal configured to generate a control signal for switching the iontophoresis mode or controlling the intensity of the electrical signal and transmit the control signal to the drug delivery device to control the drug delivery to the skin.

8. The iontophoresis transdermal drug delivery system of claim 7, wherein the mobile terminal switches the polarity of the electrical signal according to the control signal.

9. The iontophoresis transdermal drug delivery system of claim 7, wherein the iontophoresis mode includes a first mode for increasing drug permeation to the skin and a second mode for interfering with drug permeation to the skin.

10. The iontophoresis transdermal drug delivery system of claim 8, wherein when the polarity of the drug is anionic, the mobile terminal switches the first mode to an anodic mode where the polarity of the electrical signal is anodic, and switches the second mode to a cathodic mode where the polarity of the electrical signal is cathodic.

11. The iontophoresis transdermal drug delivery system of claim 8, wherein when the polarity of the drug is cationic, the mobile terminal switches the first mode to a cathodic mode where the polarity of the electrical signal is cathodic, and switches the second mode to an anodic mode where the polarity of the electrical signal is anodic.
